(19) United States
(12) Patent Application Publication
(10) Pub. No.: US 2020/0261171 A1
(43) Pub. Date: Aug. 20, 2020

(54) ROBOTIC SYSTEM WITH ARTICULATING PROBE AND ARTICULATING CAMERA

(71) Applicant: MEDROBOTICS CORPORATION, Raynham, MA (US)

(72) Inventors: Thomas Calef, Bridgewater, MA (US); Richard Andrews, North Attleboro, MA (US); Jesse Mitchell, Jamaica Plain, MA (US); R. Maxwell Flaherty, Topsfield, MA (US); J. Christopher Flaherty, Auburndale, FL (US)

(21) Appl. No.: 16/761,522

(22) PCT Filed: Nov. 6, 2018
(86) PCT No.: PCT/US18/59338

§ 371 Date: May 5, 2020

Related U.S. Application Data
(60) Provisional application No. 62/614,346, filed on Jan. 5, 2018.
(60) Provisional application No. 62/582,283, filed on Nov. 6, 2017.

(51) Int. Cl. A61B 34/30; A61B 1/00; A61B 1/008; A61B 1/018

(52) U.S. Cl. CPC A61B 34/30; A61B 1/00149; A61B 2034/301; A61B 1/018; A61B 1/008

(57) ABSTRACT

A system for performing a medical procedure on a patient includes an articulating probe assembly. At least one tool is configured to translate through one of the at least two working channels of the probe assembly. A camera device includes a shaft comprising an articulating distal portion with a distal end and a camera assembly positioned on the distal end of the shaft. The system is configured to articulate the shaft distal portion to position the camera distal assembly outside of the probe assembly.

### RELATED APPLICATIONS

This application claims the benefit of U.S. Provisional Application No. 62/582,283, filed Nov. 6, 2017, the content of which is incorporated herein by reference in its entirety.

This application claims the benefit of U.S. Provisional Application No. 62/614,346, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2017/042449, filed Jul. 17, 2018, PCT Publication No. WO2018/xxxxxx, the content of which is incorporated herein by reference in its entirety.

This application is related to U.S. Provisional Application No. 62/613,899, filed Jan. 5, 2018, the content of which is incorporated herein by reference in its entirety.

This application is related to U.S. Provisional Application No. 62/614,223, filed Jan. 5, 2018, the content of which is incorporated herein by reference in its entirety.

This application is related to U.S. Design application Ser. No. 29/632,148, filed Jan. 5, 2018, the content of which is incorporated herein by reference in its entirety.

This application is related to U.S. Provisional Application No. 62/614,224, filed Jan. 5, 2018, the content of which is incorporated herein by reference in its entirety.

This application is related to U.S. Provisional Application No. 62/614,228, filed Jan. 5, 2018, the content of which is incorporated herein by reference in its entirety.

This application is related to U.S. Provisional Application No. 62/614,225, filed Jan. 5, 2018, the content of which is incorporated herein by reference in its entirety.

This application is related to U.S. Provisional Application No. 62/614,240, filed Jan. 5, 2018, the content of which is incorporated herein by reference in its entirety.

This application is related to U.S. Provisional Application No. 62/614,235, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### FIELD

The present inventive concepts generally relate to the field of surgical instruments, and more particularly, to articulated probe assemblies, systems and methods incorporating the same, and systems and methods for performing a surgical procedure.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

According to an aspect of the present inventive concepts, a system for performing a medical procedure on a patient, comprising: an articulating probe assembly, comprising: an inner probe comprising multiple articulating inner links; an outer probe surrounding the inner probe and comprising multiple articulating outer links; a probe distal portion including an outer surface, distal end, and a central axis, wherein the outer surface defines a probe projection extending distally from the probe assembly and along the central axis; and at least two working channels that exit the probe distal end; at least one tool configured to translate through one of the at least two working channels, and a camera device, comprising: a shaft comprising an articulating distal portion with a distal end; and a camera assembly positioned on the distal end of the shaft; wherein the system is configured to articulate the shaft distal portion to position the camera distal assembly outside of the probe projection.

In some embodiments, the medical procedure comprises a trans-vaginal procedure.

In some embodiments, the medical procedure comprises a trans-abdominal procedure.

In some embodiments, the probe distal portion comprises multiple links.

In some embodiments, the probe distal portion comprises a wrist portion. The probe distal portion can further comprise an elbow portion.

In some embodiments, the camera assembly includes a CCD camera.

In some embodiments, the camera device includes an optical fiber and the camera assembly includes a lens optically connected to the optical fiber.

In some embodiments, the camera device comprises a 3D camera.

In some embodiments, the articulating distal portion of the camera device shaft articulates distal to the distal end of the probe.

In some embodiments, the probe distal portion comprises a slot, and the articulating distal portion of the camera device shaft articulates through the slot.

In some embodiments, the system is configured to rotate the camera assembly to an outwardly rotated position. The system can further comprise a control which when activated causes the rotation of the camera assembly. Activation of the control can cause the inner probe and the outer probe to lock. Activation of the control can cause the at least one tool to transition to an unlocked state. Activation of the control can cause an image provided by the system to rotate. Activation of the control can cause an image calibration procedure to be performed. The system can further comprise at least one control configured to alternatingly control both the probe assembly and the at least one tool, and the system can be configured to rotate the camera assembly when the control changes from controlling the probe to controlling the at least one tool. The system can be further configured to automatically rotate the camera assembly to an outwardly rotated position. The system can further comprise a control to advance the at least one tool, and the automatic rotation occurs when the control can be activated. The automatic rotation can occur when the system transitions to a state in which the inner probe can be locked and the outer probe can be locked.

In some embodiments, the system is configured to prevent advancement of the at least one tool if the camera assembly is not in an outwardly rotated position.

In some embodiments, the medical procedure is performed at a target location comprising an equivalent diameter, and the probe comprises an outer diameter, and the target location diameter is at least two times, at least three times, at least four times the outer diameter of the probe.

In some embodiments, one of the inner probe or the outer probe is configured to transition between a rigid mode and a flexible mode, and the other of the inner probe or the outer probe is configured to transition between a rigid mode and a flexible mode and to be steered. The outer probe can be configured to be steered.

In some embodiments, the articulating probe is constructed and arranged to be inserted into a natural orifice of the patient. The natural orifice can comprise at least one of the anus, vagina, meatus, nostril, mouth, ear, and combinations thereof.

In some embodiments, the articulating probe is constructed and arranged to be inserted through an incision in the patient.

In some embodiments, the at least one tool comprises a flexible tool.

In some embodiments, the at least one tool comprises a rigid tool.

In some embodiments, the at least one tool comprises at least one of a laser delivery element, scissor, blade, cautery element, suction element, irrigation element, grasper, surgical stapler or other tissue securing element, and combinations thereof.

According to another aspect of the present inventive concepts, a method of operating an articulating probe, wherein the articulating probe is advanced through a luminal pathway, the method comprising: introducing the articulating probe proximate a proximal end of a luminal pathway, the articulating probe comprising an inner and an outer probe and wherein the inner and outer probes each include a plurality of links with a distal-most link; the articulating probe further comprising a camera device including a shaft and a camera assembly on the distal end of the shaft; and advancing both the inner and outer probes of the articulating probe in a double limp mode, wherein the inner and outer probe simultaneously advance each in a limp state within the luminal pathway; transitioning the inner probe from the limp state to a rigid state; continue advancing the articulating probe in a follow the leader mode, comprising: advancing the limp outer probe such that its distal-most link extends at least one link's length beyond the distal-most link of the inner probe, wherein the rigid inner probe is not advanced; transitioning the outer probe from the limp state to a rigid state and the inner probe from the rigid state to the limp state; and advancing the limp inner probe such that its distal-most link is coextensive with the distal-most link of the outer probe, wherein the rigid outer probe is not advanced; and wherein the inner and outer probes are alternatingly advanced to approach at least one target location within the luminal pathway, and wherein the inner and/or outer probes are steerable in the limp state; rotating the camera assembly to an outwardly rotated position; performing at least one procedure at the at least one target location; and retracting the articulating probe from the luminal pathway such that both the inner and outer probes are retracted in the double limp mode and/or the follow the leader mode.

In some embodiments, the limp outer probe can be advanced such that its distal-most link extends between two and twenty link's length beyond the distal-most link of the inner probe.

In some embodiments, the method further comprises articulating the shaft of the camera device to reposition the camera assembly.

In some embodiments, the articulating probe includes a slotted distal portion, and the shaft articulates through the slot.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of a system for performing a medical procedure, consistent with the present inventive concepts.

FIGS. 2A-C illustrate graphic demonstrations of an articulating probe, consistent with the present inventive concepts.

FIGS. 3A and B illustrate schematic views of the distal portion of an articulated probe including a camera device and a tool, consistent with the present inventive concepts.

FIGS. 4A and B illustrate schematic views of the distal portion of an articulated probe including a slotted distal portion, a camera device, and a tool, consistent with the present inventive concepts.

FIGS. 5A-D illustrate a sequence of schematic drawings of advancing a camera device from a confined space, consistent with the present inventive concepts.

FIG. 6 illustrates a perspective view of a probe system including a camera device and multiple tools in a triangulated position, consistent with the present inventive concepts.

FIGS. 7A and 7B illustrate perspective views of a camera from the front and back, consistent with the present inventive concepts.

FIGS. 8A and 8B illustrate perspective views of a camera operably attached to a surgical tool, consistent with the present inventive concepts.

FIG. 9A illustrates a perspective view of a camera connector, consistent with the present inventive concepts.

FIGS. 9B-D illustrate perspective views of a camera cable adaptor, consistent with the present inventive concepts.

FIGS. 10A and 10B illustrate a perspective view and a sectional perspective view of a distal link, respectively, consistent with the present inventive concepts.

### DETAILED DESCRIPTION

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems for performing a medical procedure, such as a trans-vaginal or trans-abdominal medical procedure performed at a target location within a patient. The systems include an articulating probe assembly, at least one tool, and a camera device that includes a shaft with a distal end, and a camera assembly on the distal end of the shaft. The articulating probe assembly comprises an inner probe comprising multiple articulating inner links; an outer probe surrounding the inner probe and comprising multiple articulating outer links; and a probe distal portion including an outer surface, distal end, and a central axis. The outer surface defines a probe projection extending distally from the probe assembly and along the central axis. The probe assembly can further include one or more working channels that slidingly receive one or more tools, and/or at least one working channel that slidingly surrounds the shaft of the camera device. The shaft of the camera device can include one or more articulating portions, such as portions including articulatable links. The system is configured to allow an operator to articulate the shaft of the camera device to position the camera distal assembly outside of the probe projection. In some embodiments, the probe assembly includes a slot extending proximally from the distal end of the probe assembly, and the shaft of the camera device can be articulated through the slot.

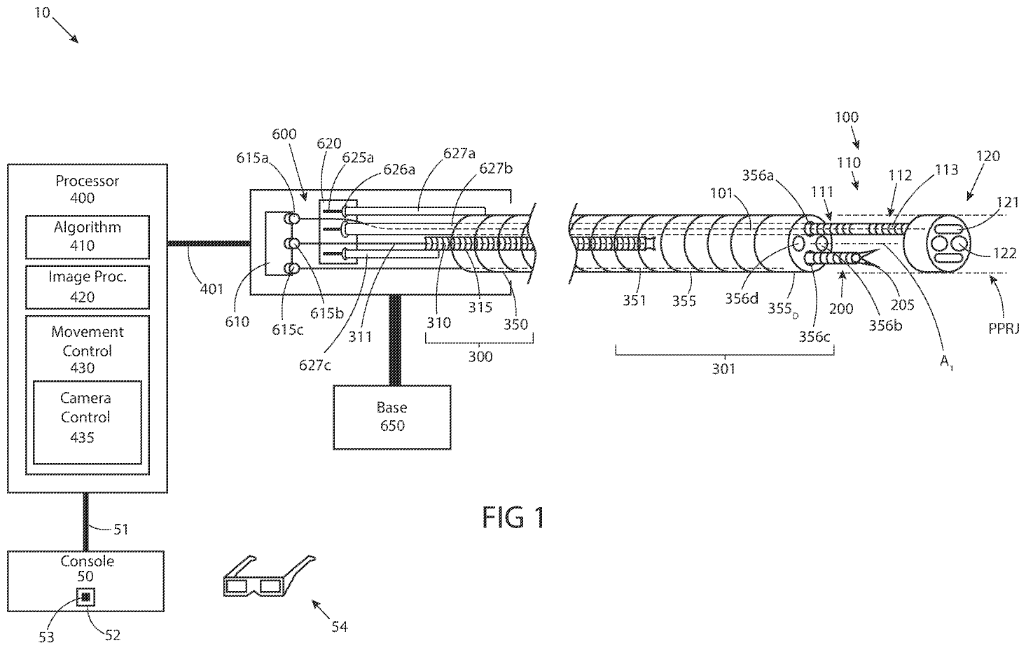

Referring now to FIG. 1, a schematic view of a system for performing a medical procedure is illustrated, consistent with the present inventive concepts. System 10 includes an articulating probe assembly, probe 300, one or more tools, such as tool 200 shown, and a camera device, camera 100. System 10 can be used to perform a surgical procedure at a target location within a patient's body cavity. The target location can comprise a location within: the colon; the intestine; the esophagus; a nasal passageway; the vaginal canal; an ear canal; an artificially insufflated cavity in the abdomen; and/or other body lumen or body location. Additionally, the target location can comprise a 3D space comprising an equivalent diameter as defined herein. In some embodiments, system 10 provides access to the target location via an orifice selected from the group consisting of: the anus; the vagina; the meatus (distal end of the urethra); a nostril; the mouth; an ear; a surgical incision; and combinations of one or more of these.

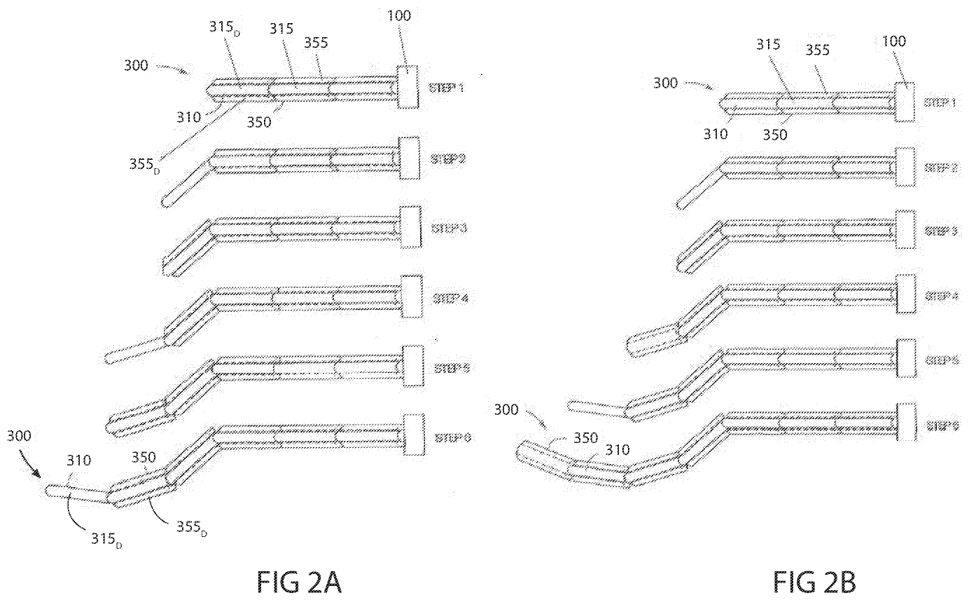

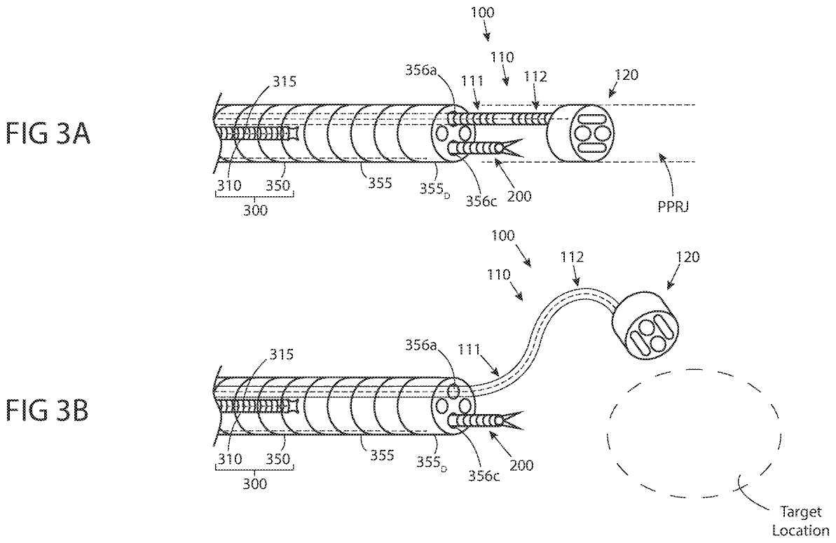

Probe 300 includes an inner probe 310 comprising multiple inner links 315 and an outer probe 350 comprising multiple outer links 355 and slidingly surrounding inner probe 310. Probe 300 includes a distal portion 301 configured to be steered. Distal portion 301 can comprise an outer diameter. In some embodiments, the target location comprises a diameter at least two times, at least three times, or at least four times the outer diameter of distal portion 301. In some embodiments, distal portion 301 comprises a slot 3561, as described herebelow in reference to FIGS. 4A-B. Articulating probe 300 can be constructed and arranged as is described in reference to applicant's co-pending U.S. patent application Ser. No. 15/378,723, filed Dec. 14, 2016, the contents of which are incorporated herein by reference in their entirety for all purposes, and/or as described in further detail herebelow in reference to FIGS. 2A-C. Outer probe 350 can comprise one or more steering cables, cables 351, configured to steer outer probe 350, and/or transition outer probe 350 between limp and rigid states, as described herein. Inner probe 310 can comprise one or more steering cables, cables 311, also configured to steer inner probe 310 and/or transition inner probe 310 between limp and rigid states. In some embodiments, only one of outer probe 350 or inner probe 310 is steered, while both can transition between limp and rigid states. Probe 300 includes at least two working channels, such as channels 356a-d shown (generally channels 356). Channels 356 can comprise channels positioned between mating recesses between the outer surface of inner probe 310 and the inner surface of outer probe 350.

Distal portion 301 of outer probe 300 can comprise at least a distal most link, distal link 355. Distal portion 301 includes an outer surface, and a central axis Ai extending along the path of probe 300 proximal to distal link 355, and normal to the distal face of distal link 355D distally. The outer surface, projected distally along this axis defines a “probe projection”, PPRJ, extending beyond the distal end of probe 300.

Tool 200 comprises one or more tools that can be inserted into and translated within channels 356. Tool 200 can comprise flexible tools and/or rigid tools with end effectors 205. Tool 200 can comprise one, two, three, or more tools selected from the group consisting of: a laser delivery element; a scissor; a blade; a cautery element; a suction element (e.g. a nozzle operably connected to a vacuum source); an irrigation element (e.g. a nozzle operably connected to an irrigation source); a grasper; a surgical stapler or other tissue securing element; and combinations thereof.

Camera device 100 comprises an elongate device including shaft 110 and camera assembly 120 positioned on the distal end of shaft 110. In some embodiments, camera device 100 comprises a 3-D camera. Shaft 110 comprises multiple links 113 configured to articulate relative to each other, via articulation caused by tensioning of one or more cables, cable 101 shown. Shaft 110 can include one, two, three, or more independently articulatable segments, such as to enable a “wrist and elbow” type configuration, allowing articulation of camera device 100 in separate, independent directions. For example, shaft 110 can include two independently articulatable segments, articulating portion 111, comprising multiple links 113, and articulating portion 112, comprising multiple links 113. In some embodiments, first articulating portion 111 can be constructed and arranged to articulate in a single degree of freedom, and second articulating portion 112 can be constructed and arranged to articulate in two degrees of freedom. In some embodiments, links 113 comprise hinged links that alternate in alternating directions from link to link (e.g. 90° alternating links, such that three consecutive links enable articulation in 2 degrees of freedom). In some embodiments, links 113 of first articulating portion 111 do not alternate, and only articulate in a single direction, enabling a tighter radius of curvature for articulating portion 111 (e.g. tighter than that of articulating section 112). Camera assembly 120 can include one or more light capturing elements, lenses 122, and one or more illumination sources, lights 121. Lens 122 can direct light from outside camera assembly 120 to one or more sensors (e.g. CCD sensors) within camera assembly 120 and/or into one or more optical fibers, such as an optical fiber positioned through camera assembly 100 and optically attached to one or more sensors proximal to camera assembly 120 (e.g. imaging sensor positioned outside of probe 300). Camera assembly 120 can be constructed and arranged as is described in reference to applicant's co-pending International PCT Patent Application Serial Number PCT/US2017/054297, titled “Optical Systems For Surgical Probes, Systems And Methods Incorporating The Same, And Methods For Performing Surgical Procedures”, filed Sep. 29, 2017, the content of which is incorporated herein in its entirety for all purposes. Additionally or alternatively, camera assembly 120 can be of similar construction and arrangement to the similar devices described in applicant's co-pending application U.S. Provisional Application No. 62/614,225, filed Jan. 5, 2018, “Robotically Controlled Surgical Tool”, by Mitchell, et al., the content of which is incorporated herein by reference in its entirety.

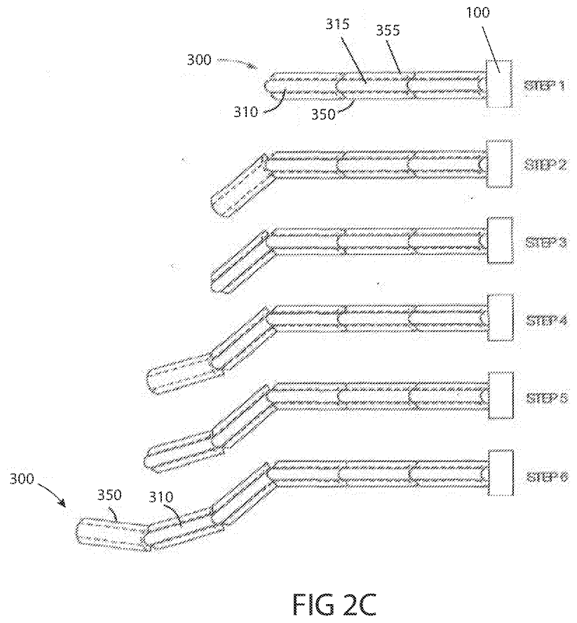

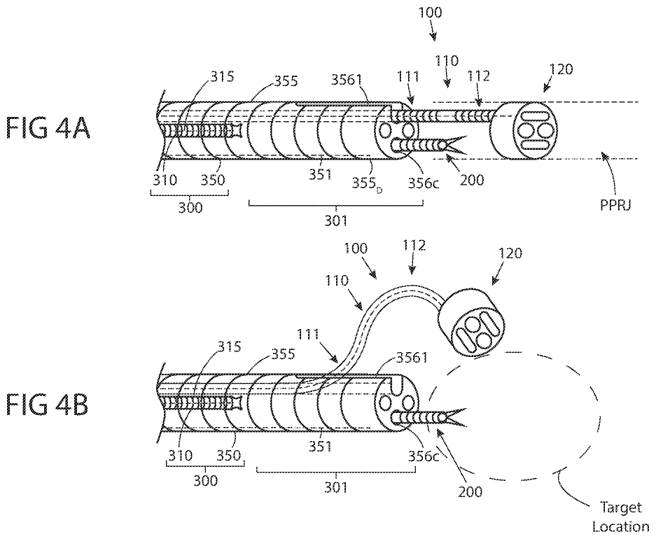

Shaft 110 of camera device 100 can be slidingly positioned within and exiting the distal end of a channel 356 (channel 356a shown) of probe 300, with camera assembly 120 positioned distal to the distal end of probe 300 (e.g. all articulation of camera device 100 is beyond the distal end of probe 300). In a first orientation, camera assembly 120 can be positioned within the region PPRJ of probe 300, such that camera assembly 120 is aligned with the distal portion 301 of probe 300. Additionally, camera assembly 120 can be positioned close to and/or abutting distal link 355D, relatively covering the distal end of one or more working channels 356 (e.g. in that alignment preventing one or more tools 200 from exiting the distal end of working channels 356). Camera device 100 can be advanced, such that camera assembly 120 moves distally from distal link 355D, allowing one or more tools 200 to exit working channels 356, as shown in FIGS. 3A-B and 4A-B. In some embodiments, shaft 110 can operably attach to camera assembly 120 offset from the center axis of camera assembly 120. In these embodiments, camera device 100, or at least camera assembly 120, can be rotated to an outwardly rotated position, such as a rotation of approximately 180°, such as to reposition camera assembly 120 outside of the region PPRJ of probe 300, providing clearance for one or more tools 200 to exit working channels 356, as shown in FIGS. 5A-D.

System 10 can be configured to rotate camera assembly 120 to the outwardly rotated position via a control 53 of user interface 52. Control 53 can be configured to cause at least one of the following to occur in addition to rotating camera assembly 120: lock inner probe 310; lock outer probe 350; lock inner probe 310 and outer probe 350; transition tool 200 to an unlocked state; rotate an image provided by system 10, such as to adjust for the camera rotation; perform an image calibration procedure; and combinations thereof.

In some embodiments, system 10 is configured to automatically rotate camera assembly 120 to the outwardly rotated position. Control 53 can be configured to alternatingly control both probe 300 and a tool 200, such that control 53 rotates camera assembly 120 automatically when control 53 changes from controlling probe 300 to controlling a tool 200. In some embodiments, control 53 can advance tool 200 such that before tool 200 is advanced beyond the distal end of probe 300, camera assembly 120 is rotated automatically (e.g. automatic rotation of camera assembly 120 prior to the advancement of tool 200 beyond the distal end of probe 300). System 10 can be configured to automatically rotate camera assembly 120 to the outwardly rotated position when system 10 transitions to a state in which inner probe 310 is locked and outer probe 350 is locked (e.g. either or both probes 310, 350 is locked and the other transitions to locked, or both probes 310, 350 transition to locked). System 10 can be configured to prevent advances of tool 200 if camera assembly 120 is not in the outwardly rotated position.

In some embodiments, the camera position is robotically controlled independent of the probe and instrument positions, such as to allow the user to choose optimal viewing angle for a given procedure and set of instruments. In some embodiments, the camera can be positioned to view instruments as they are introduced into the surgical field, and then repositioned to follow the instruments to the surgical site. As the needs of the user change during the procedure, the camera can be repositioned accordingly.

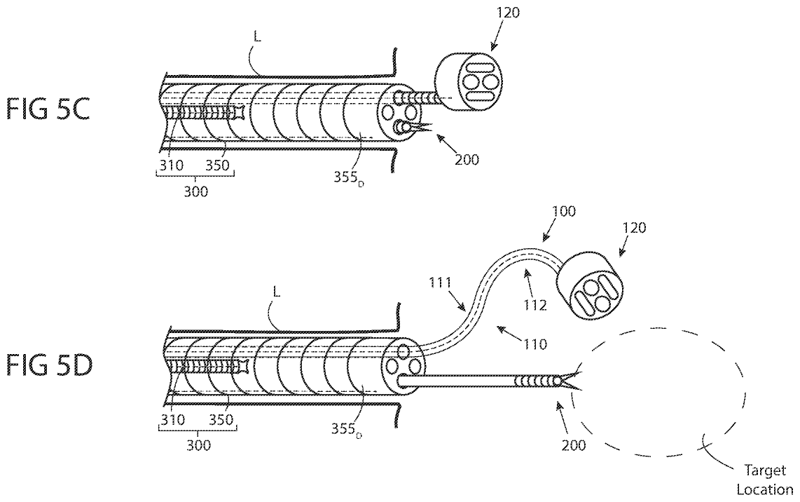

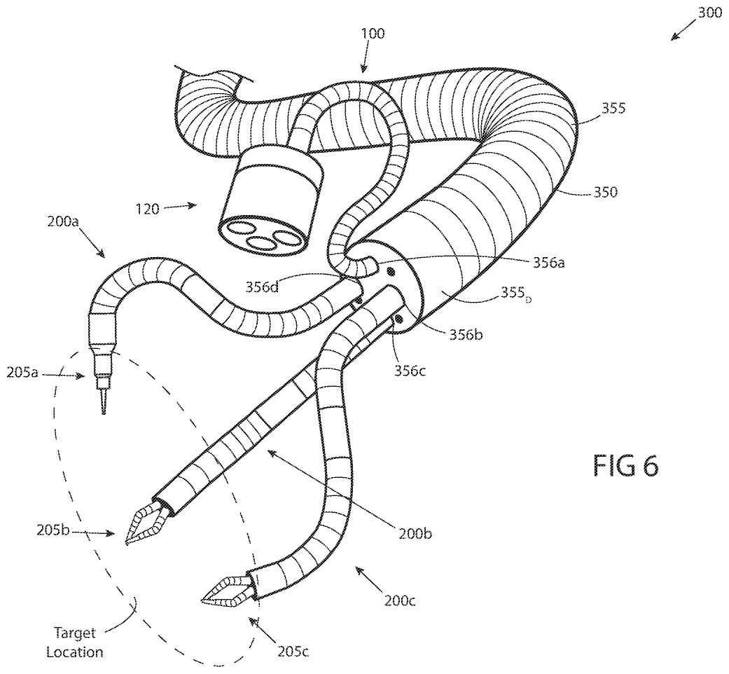

In some embodiments, the camera can be detached from a robotic instrument at distal tip, such as to allow for camera sterilization separate from an instrument, and to allow for a replacement of damaged cameras or instruments. In FIGS. 7A-B, the camera assembly (including a camera cable) is shown removed from a robotic instrument. In FIGS. 8A-B, the camera assembly is shown attached to a robotic instrument (e.g. with the camera cable extending through the instrument.

In some embodiments, the camera attachment point is positioned on a centerline of the housing, at a top edge of proximal cap, such that: the cable can be routed through the attachment point (distal link) and center lumen of the camera instrument; there is no need for exterior routing along probe path (e.g. it does not impact probe OD); the camera can be pivoted along its centerline; the camera can be positioned within the OD of the probe during probe introduction, and then pivoted 180° to allow instruments to be introduced through their channels; and the camera can achieve a steeper viewing angle when visualizing instrument introduction (e.g. to reduce the “blind spot” at the distal tip and allow the user to visualize the instruments sooner as they are introduced). The camera attachment point is illustrated in FIGS. 7A-8B

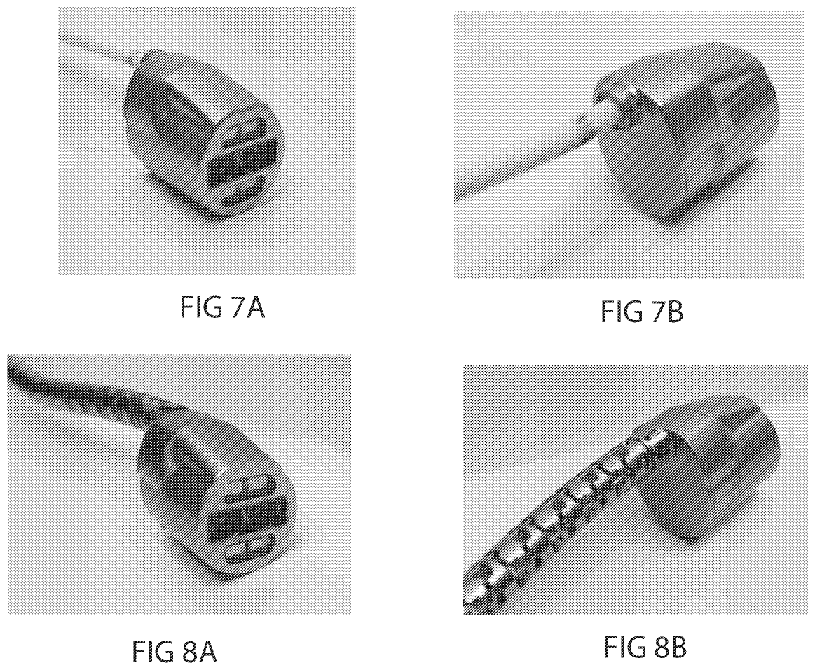

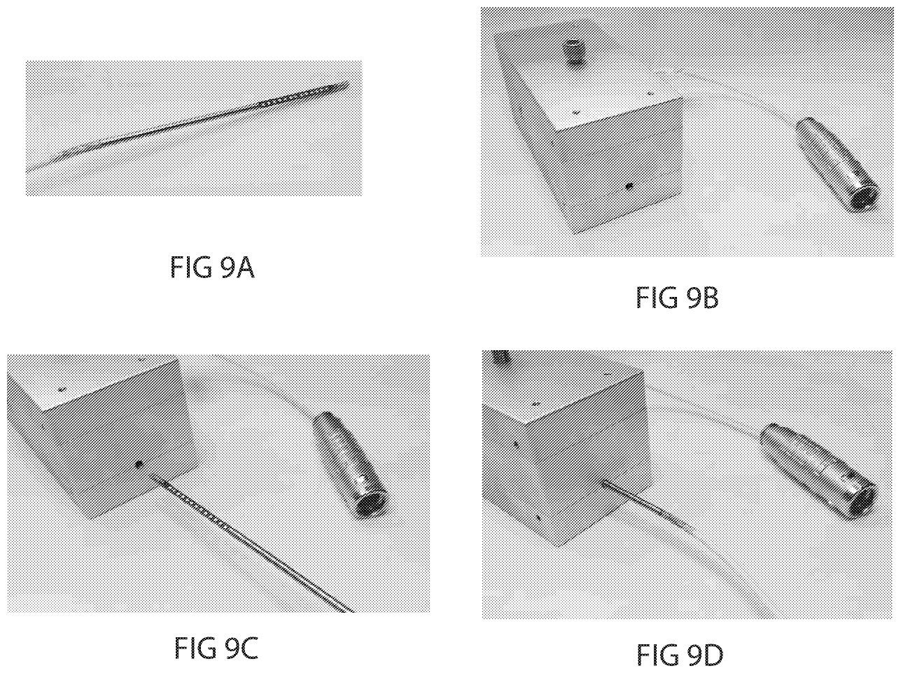

In some embodiments, a custom camera connector has an OD of approximately 0.105 inches, such that it can travel through the center lumen of the camera instrument. In some embodiments, the camera can be installed and removed from the instrument without cutting off the connector and re-soldering. The connector can comprise a long, skinny PCB potted inside of a stainless-steel tube, with surface pads exposed for pin engagement, as shown in FIG. 9A. The camera cable conductors can be soldered to the PCB and potted inside of the stainless-steel tube. The tip of the connector can be keyed for proper installation into an adapter. The tip of the connector can comprise female threads to accept a pull tool (e.g. a steel cable with male fine thread tip) and be pulled through the instrument and probe. In some embodiments, the camera can receive power and transmit data through the instrument and instrument drive, eliminating the need for an external connector.

In some embodiments, the camera connector plugs into a custom adapter unit which outputs to a standard connector (e.g. a Lemo connector), as shown in FIGS. 9B-D. The adapter can comprise an aluminum housing with a cylindrical channel to accept the camera connector. The connector can be inserted until it comes to a hard stop within the adapter and is keyed in the correct position via a feature at its tip. Once the connector is fully inserted, the user can tighten a screw through the top face of the adapter which lowers a spring-loaded PCB insert. One or more spring pins on the PCB insert can contact the connector pads as the insert is lowered to its locked position. The PCB can be wired to a short section of camera cable which exits the adapter and terminates to a Lemo or other standard or custom camera connector.

In some embodiments, eleven links in the proximal joint and the first three links in the distal articulations section of the camera instrument are uni-directional, such that they articulate only on one axis. The uni-directional links can achieve a tighter radius along their articulation section versus bi-directional links. A tighter radius can allow for the camera to move up and away from the probe in a shorter length than bi-directional links would allow. In some embodiments, an articulation section using uni-directional links articulates “up” and “down”, but not “left” and “right” (e.g. relative to the orientation of the camera).

In some embodiments, the proximal cap of the camera includes rectangular slots machined into the steel around the cable exit to allow the “fingers” of the distal link to engage. The fingers of the distal link can create a mechanical connection between the camera instrument and the camera, allowing for linear and/or rotational motion.

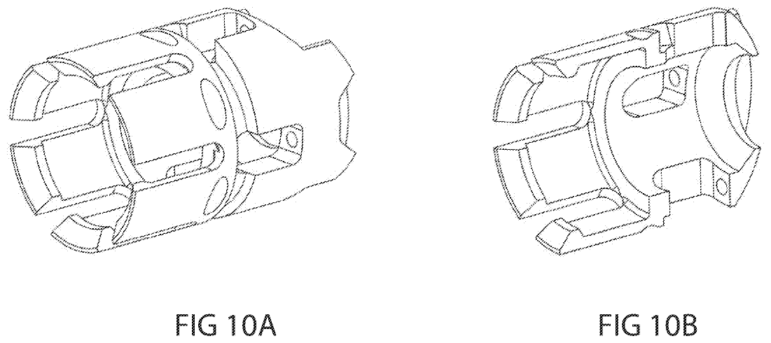

In some embodiments, the distal link of the camera instrument includes chamfers on its “fingers” to allow the camera to be removed from the instrument. The distal link can include a slot to accept the tang on the instrument collar, which is used to “lock” the fingers down while the camera is installed. The camera can be removed from the distal link by rotating the collar, thereby releasing the tang from the slot, and pushing the collar proximally. The distal link “fingers” can be splayed open using a small tool to disengage from the proximal cap of the camera. The distal link is illustrated in FIGS. 10A-B.

System 10 includes a manipulation assembly 600 configured to operably attach to a base unit 650 and articulating probe 300. Manipulation assembly 600 can comprise a cable control assembly 610 and a position control assembly 620. Cable control assembly 610 is configured to manipulate the tension of one or more steering cables, for example cables 311, 351, and/or 101 described hereabove, to articulate (e.g. steer) and/or control the rigidity of inner probe 310, outer probe 351, and/or camera device 100, respectively (e.g. transition each of these between limp and rigid states). Cable control assembly 610 can comprise one or more tensioning mechanisms, such as bobbins 615a-c shown, onto which one or more steering cables can be wound. Each bobbin 615 can include or otherwise be engaged with a rotational element (e.g. a motor) configured to rotate the associated bobbin 615 in a clockwise and/or counter-clockwise direction, such as to tension or de-tension the associated cable (e.g. to steer and/or transition between the limp and rigid states). In some embodiments, one or more tools 200 can operably attach to cable control assembly 610, such that cable control assembly 610 can robotically manipulate the attached tool 200. Position control assembly 620 is configured to manipulate the rotation and/or linear position of inner probe 310, outer probe 350, a portion of camera device 100, and/or another tool of system 10. Position control assembly 620 can comprise one or more connector assemblies, such as connector assemblies 627a-c (generally connector assemblies 627) shown, operably attached to outer probe 350, camera device 100, and inner probe 310, respectively. Connector assemblies 627 can operably attach to linear controls 625 configured to adjust the linear position of the attached device relative to base unit 650. Additionally or alternatively, connector assemblies 627 can operably attach to rotational controls 626 configured to rotate the attached device, and/or a portion thereof (e.g. rotate an inner portion and/or the distal portion of an attached device).

System 10 further comprises a processor 400, including one or more algorithms, algorithm 410, for execution by processor 400, which enable the operation of system 10. Processor 400 can further include an imaging processing unit 420 for receiving and processing optical signals (e.g. signals received from camera device 100), and/or other image data. Processor 400 is operably attached to manipulation assembly 600, camera device 100, and/or other electronic components of system 10 via one or more data and or power transmission conduits, for example cable 401 shown. In some embodiments, processor 400 communicates wirelessly with one or more components of system 10. System 10 can further comprise a console 50. Console 50 can comprise a user interface 52 configured to receive commands from a surgeon, technician, and/or other operator of system 10. User interface 52 can include one or more user input devices, such as a joystick, a multi axis input device, a mouse, a keyboard, and/or a touchscreen device. User interface 52 can further include one or more output devices, such as a monitor, speaker, haptic controller, and/or an indicator light. In some embodiments, console 50 includes one or more conduits 51 (e.g. wires, optical fibers, fluid tubes, and the like) configured to operably attach console 50 to processor 400.

In some embodiments, user interface 52 includes a 3D monitor, such as a 3D monitor configured to display stereoscopic images to a user, such that the user can perceive a 3D image (or images, such as video). In these embodiments, system 10 can include glasses 54, such as polarized glasses configured to allow the user to perceive the 3D image. In some embodiments, glasses 54 further comprise lenses configured to provide protection from laser or other energy which may be harmful to the eye.

Processor 400 can further include a movement control unit 430, further comprising a camera control unit 435. Movement control unit 430 can receive one or more commands from console 50 (e.g. user input commands for the steering of probe 300 and/or camera device 100), and provide operational commands to manipulation assembly 600 to operably control an attached device.

Referring now to FIGS. 2A-C, graphic demonstrations of an articulating probe are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 2A-C show the concept of how different embodiments of articulating probe 300 operate. Referring to FIG. 2A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 2A. Now, inner probe 310 is pushed forward by feeder 100, and a distal-most inner link 315D is steered, as seen in step 2 in FIG. 2A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 2A. Now, outer probe 350 is made rigid, inner probe 310 limp, and the procedure then repeats. One variation of this approach is to have outer probe 350 be steerable as well. The operation of such a device is illustrated in FIG. 2B. In FIG. 2B it is seen that each mechanism is capable of catching up to the other and then advancing one link beyond. According to one embodiment, outer probe 350 is steerable and inner probe 310 is not. The operation of such a device is shown in FIG. 2C.

In medical applications, operation, procedures, and so on, once articulating probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of articulating probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within articulating probe 300.

Inner probe 310 and/or outer probe 350 are steerable and inner probe 310 and outer probe 350 can each be made both rigid and limp, allowing articulating probe 300 to drive anywhere in three-dimensions while being self-supporting. Articulating probe 300 can “remember” each of its previous configurations and for this reason, articulating probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock articulating probe 300, while inner links 315 are used to lock articulating probe 300. In a “follow the leader” fashion, while inner links 315 are locked, outer links 355 are advanced beyond the distal-most inner link 315. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of articulating probe 300. In some embodiments, the tools can be inserted through the links following positioning of articulating probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of articulating probe 300. In some embodiments, an operator can select the number of links used for steering (e.g. to select between 1 and 10 links to be advanced prior to each steering maneuver).

Referring now to FIGS. 3A and 3B, schematic views of the distal portion of an articulated probe including a camera device and a tool are illustrated, consistent with the present inventive concepts. Probe 300 includes inner and outer probes 310 and 350, respectively, and a camera device 100 that is slidingly positioned within and exiting the distal end of a working channel, channel 356a of probe 300. Camera device 100 includes camera assembly 120 positioned on the distal end of an articulatable shaft, shaft 110. Tool 200 is slidingly positioned within another working channel of probe 300, channel 356c. Tool 200 is shown exiting channel 356c for illustrative clarity, but can be positioned totally within channel 356c (e.g. during advancement of probe 300). In some embodiments, one, two or more additional tools 200 (not shown but such as are described herein) are similarly advanced through a working channel 356. Probe 300, camera device 100, tool 200, and other components of system 10 can be of similar construction and arrangement to the similar components described hereabove in reference to FIG. 1.

As shown in FIG. 3A, camera assembly 120 of camera device 100 has been advanced distally, away from distal link 355D, and tool 200 has been advanced out the distal end of channel 356c, to a location behind camera assembly 120 (advancement of tool 200 being an optional step, not necessary at this point of the procedure). Camera assembly 120 is still positioned within region PPRJ of probe 300. In FIG. 3B, articulating portion 111 of shaft 110 has been articulated in a first direction away from the central axis Ai of probe 300, and articulating portion 112 has been articulated back, towards central axis Ai, or otherwise oriented towards a target location. In this embodiment, all articulation of shaft 110 occurs distal to the distal end of probe 300. The positioning of camera assembly 120 in FIG. 3B provides an optimized view of the target location and the one or more tools 200 exiting probe 300.

Referring now to FIGS. 4A and 4B, schematic views of the distal portion of an articulated probe including a slotted distal portion, a camera device, and a tool are illustrated, consistent with the present inventive concepts. Probe 300 includes inner and outer probes 310 and 350, respectively, and a camera device 100 that is slidingly positioned within and exiting the distal end of a working channel, channel 356a of probe 300. Camera device 100 includes camera assembly 120 positioned on the distal end of an articulatable shaft, shaft 110. Tool 200 is slidingly positioned within another working channel of probe 300, channel 356c. Tool 200 is shown exiting channel 356c for illustrative clarity, but can be positioned totally within channel 356c (e.g. during advancement of probe 300). In some embodiments, one, two or more additional tools 200 (not shown but such as are described herein) are similarly advanced through a working channel 356. Probe 300, camera device 100, tool 200, and other components of system 10 can be of similar construction and arrangement to the similar components described hereabove in reference to FIG. 1.

As shown in FIG. 4A, camera assembly 120 of camera device 100 has been advanced distally, away from distal link 355D, and tool 200 has been advanced out the distal end of channel 356c, to a location behind camera assembly 120 (advancement of tool 200 being an optional step, not necessary at this point of the procedure). Camera assembly 120 is still positioned within region PPRJ of probe 300.

In some embodiments, distal portion 301 includes an elongate opening, slot 3561 as shown, between the outer surface of outer probe 350 and working channel 356a. In these embodiments, shaft 110 of camera device 100 can articulate through slot 3561, allowing articulation of shaft 110 to begin at locations proximal to the distal end of probe 300, allowing the orientation of camera assembly 120 toward target locations relatively close to the distal end of probe 300 (e.g. camera assembly 120 can image target locations closer to the distal end of probe 300 than those that could be imaged in the non-slotted configurations of FIGS. 3A-B). For example, as shown in FIG. 4B, articulating portion 111 of shaft 110 has been articulated in a first direction away from the central axis Ai of probe 300 at a location proximal to the distal end of probe 300. Articulating portion 112 has been articulated back, towards central axis Ai, or otherwise oriented towards a target location relatively proximate the distal end of probe 300. The positioning of camera assembly 120 in FIG. 4B provides an optimized view of the target location and the one or more tools 200 exiting probe 300, yet relatively close to probe 300.

Referring now to FIGS. 5A-D, a sequence of schematic drawings of advancing a camera device from a confined space are illustrated, consistent with the present inventive concepts. Probe 300 includes inner and outer probes 310 and 350, respectively, and a camera device 100 that is slidingly positioned within and exiting the distal end of a working channel, channel 356a of probe 300. Camera device 100 includes camera assembly 120 positioned on the distal end of an articulatable shaft, shaft 110. Tool 200 is slidingly positioned within another working channel of probe 300, channel 356c. In some embodiments, one, two or more additional tools 200 (not shown but such as are described herein) are similarly advanced through a working channel 356. Probe 300, camera device 100, tool 200, and other components of system 10 can be of similar construction and arrangement to the similar components described hereabove in reference to FIG. 1.

As shown in FIG. 5A, probe 300 is positioned within a body space, lumen L, with camera assembly 120 positioned proximate distal link 355D (e.g. camera assembly 120 is relatively in contact with distal link 355D), and within region PPRJ of probe 300. In FIG. 5B, probe 300 has been advanced through lumen L towards a target location, camera device 100 has been advanced away from distal link 355D, and tool 200 has been slightly advanced from channel 356c. In some embodiments, the rotation and/or positioning of camera assembly 120 (e.g. via rotation and/or articulation of shaft 110), as shown in FIGS. 5C and 5D, occurs before tool 200 (or multiple tools 200) are advanced out the distal end of probe 300 (e.g. such as when system 10 prevents advancement of a tool 200 until rotation of camera assembly 120 to an outwardly rotated position has occurred). Tool 200 is shown partially advanced in FIGS. 5B and 5C primarily for illustrative clarity. Additionally, the rotation of camera assembly 120 shown in FIG. 5C can occur in the proximal most position of camera assembly 120 shown in FIG. 5A. Again, camera assembly 120 is shown advanced in FIGS. 5B and 5C primarily for illustrative clarity.

As shown in FIG. 5C, camera assembly 120 has been rotated, to an outwardly rotated position (e.g approximately 180° as shown), out of the region PPRJ, allowing further advancement of tool 200. In some embodiments, a target location is located within or proximate region PPRJ. In these embodiments, after camera assembly 120 has rotated to allow tool 200 to exit beyond channel 356c, a surgical or other medical procedure (“surgical procedure” or “medical procedure” herein) can be performed in the configuration shown, as tool 200 extends beyond camera assembly 120 towards the target location.

Additionally or alternatively, as shown in FIG. 5D, camera device 100 can be extended and articulated, to be positioned “above” the target location, such as to provide an optimal viewing angle for the operator to perform the medical procedure.

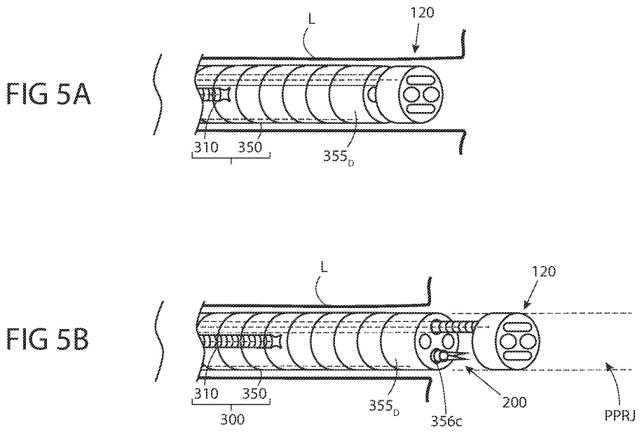

Referring now to FIG. 6, a perspective view of a probe system including a camera device and multiple tools in a triangulated position is illustrated, consistent with the present inventive concepts. Probe 300 includes inner and outer probes 310 and 350, respectively, and a camera device 100 that is slidingly positioned within and exiting the distal end of a working channel, channel 356a of probe 300. Camera device 100 includes camera assembly 120 positioned on the distal end of an articulatable shaft, shaft 110. Three tools, tools 200a-c are shown, each slidingly positioned within and exiting the distal end of a separate working channel of probe 300, channels 356b-d. In some embodiments, more or fewer tools 200 are included (such as are described herein), each tool similarly advanced through a working channel 356. Tools 200a-c can include various end effectors, such as end effectors 205a-c shown. Probe 300, camera device 100, tool 200, and other components of system 10 can be of similar construction and arrangement to the similar components described hereabove in reference to FIG. 1.

As shown in FIG. 6, probe 300 has been advanced along a tortuous, three-dimensional path, towards a target location, for example a target location to perform a surgery or other medical procedure within the body of a patient. In some embodiments, probe 300 can be advanced through a natural orifice, such as the vagina, or through a surgical incision, such as an incision into the abdomen of the patient, and advanced into a larger body cavity (e.g. larger than the opening of the orifice), such as to perform transvaginal and/or transabdominal surgery. Within this larger body cavity, camera device 100, and/or one or more tools 200 (tools 200a-c shown), can have available “space” to extend away (e.g. radially away) from the distal end of probe 300 (e.g. out of region PPRJ described herein), such as to triangulate on a target location, such that camera assembly 120 provides an optimal view of the target location, and tools 200 assume an “operator-like” anatomic position (e.g. the position of tools 200 mimic the position of the operator's hands on the controls, control 53 described herein).

Referring now to FIGS. 7A and 7B, perspective views of a camera from the front and back are illustrated, consistent with the present inventive concepts.

Referring now to FIGS. 8A and 8B, perspective views of a camera operably attached to a surgical tool are illustrated, consistent with the present inventive concepts.

Referring now to FIG. 9A, a perspective view of a camera connector is illustrated, consistent with the present inventive concepts.

Referring now to FIGS. 9B-D, perspective views of a camera cable adaptor are illustrated, consistent with the present inventive concepts.

Referring now to FIGS. 10A and 10B, a perspective view and a sectional perspective view of a distal link are illustrated, respectively, consistent with the present inventive concepts.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links;
a probe distal portion including an outer surface, distal end, and a central axis, wherein the outer surface defines a probe projection extending distally from the probe assembly and along the central axis; and
at least two working channels that exit the probe distal end,
at least one tool configured to translate through one of the at least two working channels, and
a camera device, comprising:
a shaft comprising an articulating distal portion with a distal end; and
a camera assembly positioned on the distal end of the shaft;
wherein the system is configured to articulate the shaft distal portion to position the camera distal assembly outside of the probe projection.

2.-37. (canceled)
